(19) United States
(12) Patent Application Publication
(10) Pub. No.: US 2023/0089464 A1
(43) Pub. Date: Mar. 23, 2023

(54) PREDICTIVE TEST FOR WHETHER A PATIENT WILL BENEFIT FROM PHARMACOGENOMICS TESTING

(71) Applicant: SANFORD HEALTH, Sioux Falls, SD (US)

(72) Inventors: Adam MCDERMAID, Sioux Falls, SD (US); Murat SINCAN, Sioux Falls, SD (US)

(21) Appl. No.: 17/909,152

(22) PCT Filed: Mar. 5, 2021
(86) PCT No.: PCT/US2021/020996

§ 371 Date: Sep. 2, 2022

Related U.S. Application Data
(60) Provisional application No. 62/986,065, filed on Mar. 6, 2020.

(51) Int. Cl. G16H 20/10; G16B 20/00; G16B 5/20

(52) U.S. Cl. CPC G16H 20/10; G16B 20/00; G16B 5/20

(57) ABSTRACT

Pharmacogenomic (PGx) testing provides valuable insight into patient-specific mechanisms for drug response. Limitations on the throughput of PGx testing and associated costs make it currently infeasible to test every individual, particularly in large medical enterprises such as the VA or medical centers servicing large numbers of patients simultaneously. To overcome this, a method and system is described that predicts whether a patient is likely to benefit from PGx testing. Our method permits a healthcare provider to prioritize patients for PGx testing based on which patients are identified as being most likely to benefit from the testing, and can avoid PGx testing for those patients that are likely to obtain little or no benefit from the testing, thereby saving healthcare costs.

### CROSS REFERENCE

This application claims priority to U.S. Provisional Application Ser. No. 62/986065 filed Mar. 6, 2020, incorporated by reference herein in its entirety.

### BACKGROUND

This disclosure relates to a practical, computer-implemented testing method and related system for determining, in advance, whether a particular patient would likely benefit from having a pharmacogenomics (PGx) test performed.

Pharmacogenomics is the study of how genetic variations in a person's genes affect their response to drugs. This relatively new field combines pharmacology (the science of drugs) and genomics (the study of genes and their functions) with the objective to develop and prescribe effective, safe medications and doses that will be tailored to a person's genetic makeup.

It is common knowledge that many drugs that are currently available are “one size fits all,” but in practice they don't work the same way for everyone. It can be difficult to predict who will benefit from a medication, who will not respond at all, and who will experience negative side effects.

### SUMMARY

While conducting PGx testing would inevitably benefit virtually all patients, the associated cost and throughput of PGx testing makes it infeasible to test every patient, for example all the patients currently under the care of a healthcare provider such as a hospital. Instead, a pragmatic approach would be to perform what is essentially a “pre-testing” method on patients in order to determine which patients are deemed to be most likely to benefit from the information provided by PGx testing. The system and method of this disclosure enables this approach to be realized.

In particular, while 90% of people have one or more variants in a PGx related gene, not all will actually be prescribed a medication that is PGx-relevant (i.e., those medications that have been firmly identified to have a link to a particular genetic variant). In other words, while a PGx test may identify that a patient has certain genetic variations that may predict they would benefit from certain drugs linked to such genetic variations (or have no benefit or could be harmed if they took other drugs linked to their genetic variations), in retrospect, there would little or no point in conducting the PGx test for that patient if their particular health or disease status indicates that these drugs are completely irrelevant to their particular health situation or disease state.

It is with this goal in mind that we have developed the predictive testing method and system of this disclosure, which we have called “MAPPeR” (Meaningful, Actionable

Pharmacogenomic Patient Results). MAPPeR provides a framework to determine the likelihood that a given patient will be prescribed a PGx-relevant medication, and therefore, would likely obtain benefit from a subsequent PGx test. This, in turn, results in more informed decision making for the clinical staff treating the patient and potentially providing healthcare cost savings. The method of this disclosure can also be performed widely on a set of (or all) patients at a particular healthcare provider, e.g., hospital, VA administration or subdivision thereof, and thereby enable prioritization of those patients for which for PGx testing is likely to result in substantial benefit.

In one aspect of this disclosure, a computer-implemented method for predicting whether a patient will likely benefit from a pharmacogenomics test is disclosed. The term “likely benefit from a pharmacogenomics test” means that the patient is likely to be prescribed a medication that is PGx-relevant. The method includes step (a) obtaining an input data in the form of data associated with disease status of the patient (i.e., a set of one or more ICD-10 codes, the common name of the diseases, or the equivalent), and/or data associated with a list of currently prescribed medications for the patient. This input data set could also take the form of a number, name of the patient or other information that can be linked with the patient's electronic medical record (EMR) in which case the data representing disease status and/or medications can be extracted from the EMR.

The method includes a step (b) of implementing in the computer a Bayesian network representable as a tripartite graph having links between three partitions: (1) a disease status partition having as elements representing one or more independent diseases of the patient; (2) a medications partition have as elements medications associated with the elements of the disease status partition or the medications prescribed for the patient; and (3) a genetics partition having as elements particular genetic variations which have an established pharmacogenomics relationship with one or more elements in the medications partition. The nomenclature assigned to the partitions is not particularly important, for example the genetics partition could be characterized as “biomarker”, “actionable alleles”, or the like.

The weights of links between the disease status partition and the medications partition in the Bayesian network are based on an analysis of a corpus of patient data including prescribed medications and disease diagnosis, and will have numerical values of between 0 and 1, where the higher the number the more probable the medication is prescribed for the particular disease. The weights of links between the medications partition and the genetics partition are a binary value of 1 or 0 depending on whether a pharmacogenomics relationship has been established between the elements of the medications partition and the elements of the genetics partition.

The method further includes a step (c) of generating (i.e., calculating) from the Bayesian network a probability of the patient being prescribed a medication having a pharmacogenomics relationship with one of the genetic variations in the genetics partition. This probability, referred to as P(M) in the following discussion, is based on the input data and the weights and links between the elements of the three partitions of the Bayesian network for the input data. Equation (1) below is one example of a procedure for generating the probability. A prediction of whether a patient will likely benefit from a pharmacogenomics test can be made based on the probability P(M) generated in step c). For example, a threshold may be determined based on analysis of the receiver operating characteristic curve (ROC) for the network to determine the optimum predictive performance (sensitivity and specificity). In one embodiment, the threshold is 0.01. If the probability is above the threshold the patient is recommended to have PGx testing performed.

In another aspect, an improved computer is disclosed which is configured to facilitate recommendations for conducting pharmacogenetics testing on a patient. The computer includes a) a memory storing an input data set in the form of data reflecting disease status of the patient, and/or data reflecting a list of currently prescribed medications for the patient; and (b) a processing system configured to implement a Bayesian network representable as a tripartite graph having links between three partitions: (1) a disease status partition having as elements representing one or more independent diseases of the patient; (2) a medications partition have as elements medications associated with the elements of the disease status partition or the medications prescribed for the patient; and (3) a genetics partition having as elements particular genetic variations which have a pharmacogenomics relationship with the elements in the medications partition. The weights of links between the disease status partition and the medications partition are based on an analysis of a corpus of patient data including prescribed medications and disease diagnosis, and the weights of links between the medications partition and the genetics partition are a binary value of 1 or 0 depending on whether a pharmacogenomics relationship has been established between the elements of the medications partition and the elements of the genetics partition. The computer further includes (c) executable instructions for the processing unit generating from the Bayesian network a probability of the patient being prescribed a medication having a pharmacogenomics relationship with one of the genetic variations in the genetics partition, P(M), based on the input data and the weights and links between the elements of the three partitions for the input data. A prediction of whether a patient will likely benefit from a pharmacogenomics test can be made based on the probability P(M) generated by the executable instructions c).

In another aspect, a method of selectively conducting pharmacogenomics testing on a multitude of patients of a healthcare provider (e.g., hospital, clinic, VA, etc.) includes steps of: a) entering input data for each of the multitude of patients (current diagnoses, and/or medications); b) conducting the method for predicting patient benefit of pharmacogenomics testing as explained above for each of the multitude of patients based on the input data; c) using the predictions P (M) for each of the patients to prioritize which patients should be subject to pharmacogenomics testing, and d) conducting pharmacogenomics testing for the patients prioritized in step c). Steps a), b) and c) of the method can be performed periodically, such as daily, for most or potentially all patients of the healthcare provider. The input data can optionally take the form of a patient medical record number, patient identifying information such as name, or number associated with the patient. Such information can then be linked to disease and medication data in an EMR for the patient and the input data extracted from the record and input into the Bayesian network. In one embodiment the health care provider is a hospital or medical clinic. In one possible embodiment the healthcare provider is the U.S. Veterans Administration (VA) or subdivision thereof, e.g., particular VA hospital or clinic.

### BRIEF DESCRIPTION OF THE DRAWINGS

FIG. 1 is a diagram showing an overview of the method of this disclosure.

FIG. 2 is a more detailed illustration of the Bayesian network that is used to generate the predication of whether the patient will likely benefit from PGx testing.

FIG. 3 is another illustration of the Bayesian network of FIG. 2 where the Disease Status partition includes six different diagnoses and the Medications partition includes seven different medications. The Genetics partition includes six different biomarkers.

FIG. 4 is another illustration of a portion of the Bayesian network of FIG. 2 for a simple example of two diseases in the Disease Status partition and three medications in the Medications partition, showing examples of the calculation of P(M).

FIG. 5 is a schematic illustration of the obtaining of disease status, medications, and PGx-relevant alleles from patient data and their combined use to generate a PGx testing recommendation.

FIG. 6 is a flowchart showing a method of selectively conducting pharmacogenomics testing on a multitude of patients of a healthcare provider using the testing method of this disclosure.

FIG. 7 is an illustration of a front-end user interface showing PGx testing recommendations for a set of patients of a healthcare provider, using the methodology of FIG. 6.

### DETAILED DESCRIPTION

As explained above, while pre-emptive PGx testing for all patients in a particular healthcare system could bring precision medicine at a population health level, the scarcity of resources and the need for economic efficiency calls for a tool that could stratify patients by the potential utility of this type of genetic testing. The innovation represented by this disclosure enables this ability to stratify patients for potential utility of PGx testing. It has the potential to accelerate the adoption of pharmacogenomics by increasing the yield of actionable results, which in turn could increase the engagement of clinicians and patients to this emerging field and bring the benefits of PGx testing to a much wider number of patients. Further, no blood test, genomic test, or other physically invasive test need be done to conduct the method of this disclosure, rather all is needed is information from the patient's electronic medical record.

As will be explained below, the primary end user of the system and method of this disclosure (MAPPeR for shorthand) would be a healthcare provider who could use it to identify which patients have the most immediate potential to benefit from PGx testing. Specific end users could be primary care providers at hospitals and clinics and physicians participating PHASeR programs in VA medical centers and the VA in general. PHASeR (PHarmacogenomic Action for cancer SuRvivorship) is a program funded by philanthropist Denny Sanford to provide free genetic testing for up to 250,000 veterans through a partnership between Sanford Health, the assignee of this invention, and the VA. Of course, the MAPPeR tools and methods are generally applicable to other medical systems and hospitals.

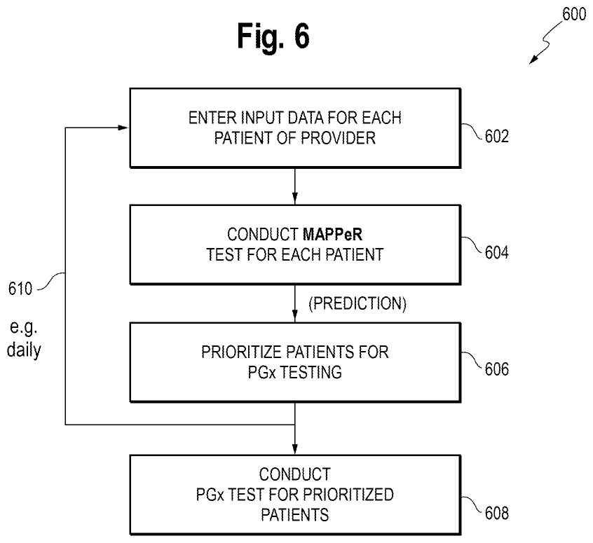

Healthcare providers could essentially match their patients from their clinic schedule each day to see whom they should recommend having PGx testing, by preforming the methods of this disclosure on a regular, e.g., daily basis, as will be described in conjunction with FIG. 6 later in this disclosure. After inputting certain clinical information or simply a patient medical record number linking to the patient's electronic medical record, MAPPeR would indicate whether an individual would be more likely to benefit from a PGx test, which may reveal genetic variants implicating drug-gene relationships across classes of medication such as opioids, anti-depressants, anti-coagulants, and other commonly prescribed drugs.

Another potential user of a version of MAPPeR could be patients themselves. An API could be set up with already available tools like the CMS Blue Button to allow patients to enter their own information via a suitable user interface (e.g. a web browser); the information entered prompts extraction of the input data needed for the Bayesian network and a prediction is generated by the Bayesian network and reported back to the patient in substantial real time. This would allow the patient to see if PGx testing would have potential benefit for them. This would empower patients to have conversations with their healthcare providers and request PGx testing.

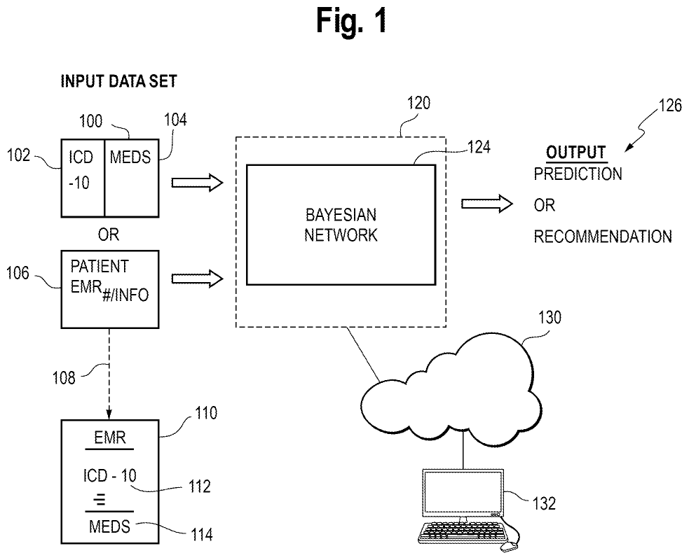

Referring now to FIG. 1, there is shown an overview of how our method works. Our method is implemented in a computer, which could take the form of a general purpose computer or network server 120, which could for example be part of the computing infrastructure of a hospital or clinic. Our method predicts whether a patient will likely benefit from a pharmacogenomics test. The method includes a step (a) of obtaining an input data set 100 in the form of data associated with disease status of the patient (such as a list 102 of ICD-10 codes, which code the diseases for which there is a diagnosis for the patient, or plain text listing the disease diagnoses), and/or data associated with a list 104 of currently prescribed medications for the patient. It will be appreciated that this input data set 100 could also take the form of patient identifying information (such as EMR number, patient name, etc.) 106 which is linked 108 to the patient's EMR 110, and in particular to the ICD-10 codes 112 and list of medications 114 in the EMR.

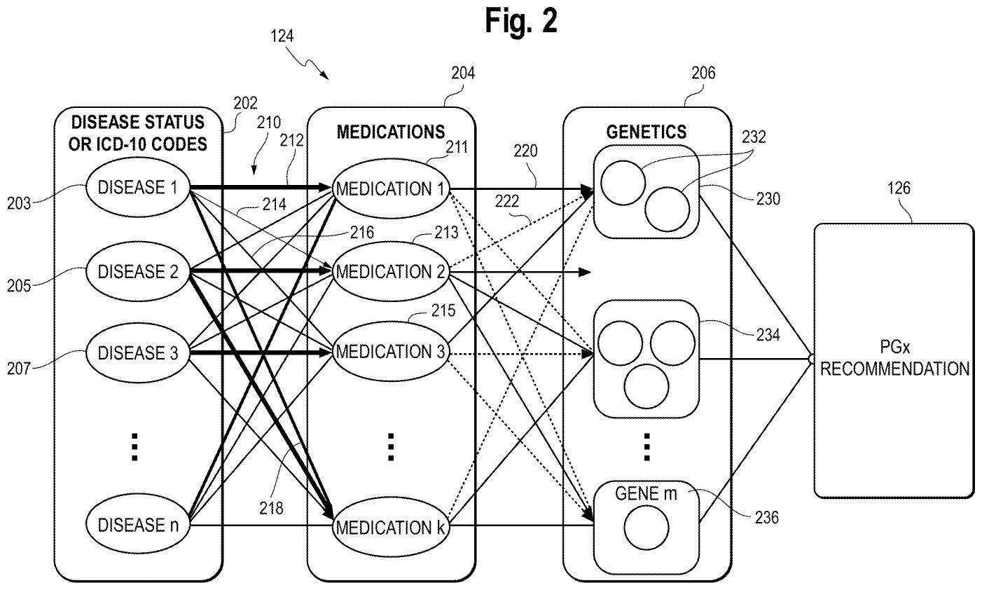

This input data set 100 is provided to the computer 120 and in particular a Bayesian network 124 is constructed and implemented in the computer. The network 123 generates an output 126 in the form of a prediction or recommendation for PGx testing as will be explained below. This Bayesian network 124 (see FIG. 2) is representable as a tripartite graph as shown in FIG. 2 having links (solid lines 210, 212, 214, 216, 218, 220, and dashed lines 222) between three partitions or groupings:

(1) a disease status partition 202 having as elements 203, 205, 207 etc. representing one or more independent diseases of the patient, there being up to N such diseases, where N is some integer greater than or equal to 1;

(2) a medications partition 204 have as elements 211, 213, 215 etc. medications that are associated with one or more of the elements 203, 205, 207 etc. of the disease status partition 202, or are medications which have been prescribed for the patient; and

(3) a genetics partition 206 (which could also be called “biomarker”, “actionable alleles” etc.) having as elements 232, 234, 236 etc. particular genes with alleles 232 (variants of the gene) which may have an established pharmacogenomics relationship with one or more the elements in the medications partition 204. It will be appreciated that as pharmacogenomics research progresses the content of the genetics partition 206 may expand over time to reflect new discoveries between genetic variants and response to pharmacological products.

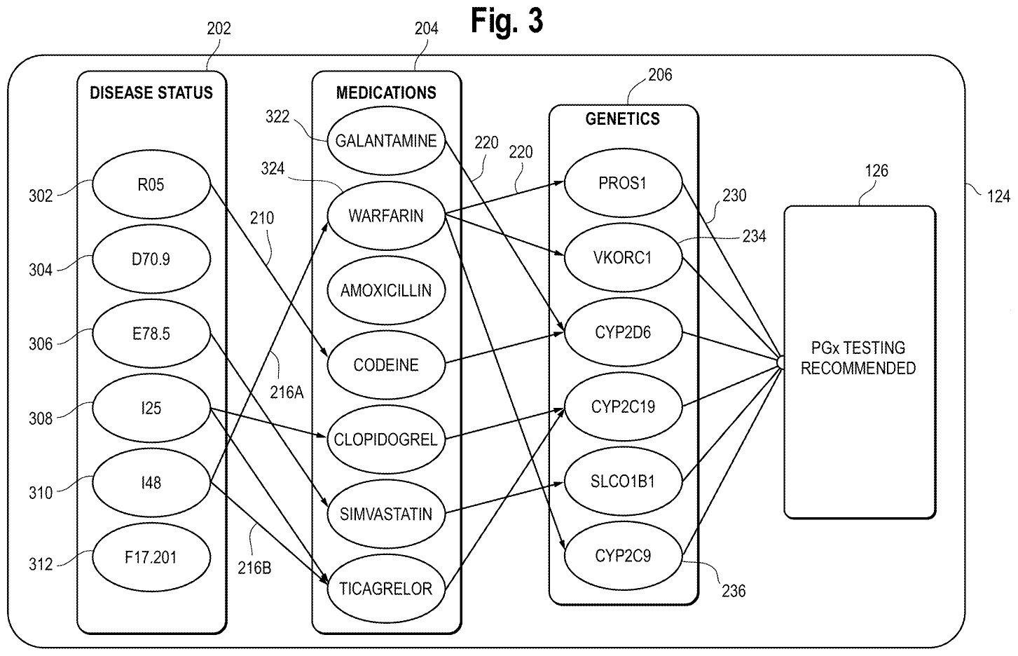

The weights of links 210, 212, 214, 216 etc. between the disease status partition 202 and the medications partition 204 are based on an analysis of a large corpus of patient data which includes prescribed medications and disease diagnosis. This will be discussed in more detail below. In FIG. 2, the links 210, 212, 214, etc. have different widths to indicate that their weight (a number between 0 and 1) is not the same, the higher the weight the thicker the line, indicating the more likely it is that a particular medication is prescribed for a particular disease diagnosis. The weights of links 220, 222 between the elements of medications partition 204 and the genetics partition 206 are a binary value of 1 or 0 depending on whether a pharmacogenomics relationship has been established between the elements of the medications partition 204 and the elements of the genetics partition 204. The links with a value of 1 are shown in solid lines such as at 200, whereas the links with value of 0 are shown as dashed lines. In FIG. 3, the links with a value of 0 are omitted.

Our method then continues with step (c) of generating (i.e., calculating) from the Bayesian network of FIG. 2 a probability of the patient being prescribed a particular medication having a pharmacogenomics relationship with one of the genetic variations in the genetics partition 206. This probability is denoted as P(M) in the discussion below. This probability is based on the input data for the patient and the weights and links between the elements of the three partitions, based on that input data. A prediction of whether a patient will likely benefit from a pharmacogenomics test can be made based on the probability P(M) generated in step c). For example, a threshold for P(M) can be established by testing the network on a patient dataset and selecting a cutoff that maximizes the sensitivity and specificity of the network. A cutoff of 0.01 is one possible threshold. If the probability P(M) is above 0.01 the patient is recommended to have the PGx test performed.

### Discussion

In our approach shown in FIG. 2, we utilize a Bayesian network model, with edges or links mapping diseases to medications (210, 212, 214, etc.) based on a conditional probability of prescription. We accessed a large corpus of de-identified patient data we had available in-house in the form of medication prescriptions and disease status for a large number of patients, to determine the weights for the links between the disease status and medication partitions of the network. The in-house data we obtained contains 3,977,249 medication prescriptions and 1,013,204 disease statuses from 741,023 unique patients between 2018 and 2019. This data was used to construct the Bayesian network model, which was then validated using the VA Precision Oncology dataset to ensure a representative and generalizable model was constructed. Overall, we were able to generate 407,321 mappings between diseases and medications, of which over 97% were identified as valid using the VA dataset.

Our method also provides a directed link from disease status to medication prescription to PGx biomarkers, as indicated by the three partitions of FIG. 2 and the binary links between the medications partition 204 and the genetics partition 206. The input for our method is data representing a set of disease diagnoses, e.g., in the form of ICD-10 codes, and/or prescribed medications. With either of these inputs provided, our Bayesian network can determine the probability that a patient would be prescribed a PGx-relevant medication in the future, thus providing evidence for the benefit of PGx testing.

The Bayesian network 124 that lies at the core of the MAPPeR program can be broken down into a tripartite graph as shown in FIG. 2. This network can then take disease status from patient medical records and assign a posterior probability of a given medication prescription. The probability of the patient being prescribed a medication M with a pharmacogenomics link to a particular genetic variant, (P (M)) can be calculated as

$$P ( {M}_{i} ) = {max}_{j} ( {G}_{i , j} ) * ( 1 - {\prod }_{k = 1}^{n} ( 1 - P ( {M}_{i} \vert {D}_{k} ) ) \qquad Equation 1$$

$$Where {G}_{i , j} = \{ \begin{matrix}1 & if Medication i and Gene j have a defined PGx link \\ 0 & otherwise\end{matrix}$$

and
where P(Mi|Dᵢ) is the weight of each link between the disease status and medications partitions.

The variable G_{i,j}, just represents the existing of a pharmacogenomics link between Gene k and Medication i. If there is a link, this value is 1; if not, it is 0. The probability will be 0 only in the event that the particular medication is not linked to any significant PGx biomarker/gene.

An assumption is built into Equation 1 that the events of being prescribed the medication given a single disease are independent (i.e. P ((M|Dᵢ)∩(M|Dⱼ))=P(M|Dᵢ)*P(M|Dⱼ)).

The links between medication and PGx biomarkers (220, 222) are binary representations of a direct connection determined by a combination of the guidelines established by the Clinical Pharmacogenetic Implementation Consortium (CPIC), see https://cpicpgx.org/guidelines/, and the FDAs Table of Pharmacogenomic Biomarkers in Drug Labeling. See gttps://www.fda.gov/drugs/science-and-research-drugs/table-pharmacogenomic-biomarkers-drug-labeling.

The probabilistic disease-to-medication mapping (represented by the links 210, 212, 214, etc. and the binary linkage from medications to PGx biomarkers (220, 222) are combined to form the core Bayesian network of the MAPPeR test method, using Equation 1 to calculate the probability P(M).

FIG. 3 is an illustration of a hypothetical Bayesian network 124 of FIG. 2 for a particular patient where the Disease Status partition 202 includes six different diagnoses 304, 304, 306, 308, 310, etc. indicated by the ICD-10 codes (RO5, D70.9, etc.) and the Medications partition 204 includes seven different medications 322, 324, etc. (Galantamine, Warfarin, etc.). The Genetics partition includes six different genes 230, 234 etc. (PROS1, VKORC1, etc.). The probability P(M) for the network shown in FIG. 3 is calculated according to Equation 1. The PGx testing recommendation 126 is based on the calculated probability and comparison to a threshold. Absence of links from disease to medication implies a probability of 0 (that medication is never prescribed for a patient who has that disease diagnosis, per our data that was used to construct the weights of the links in the network).

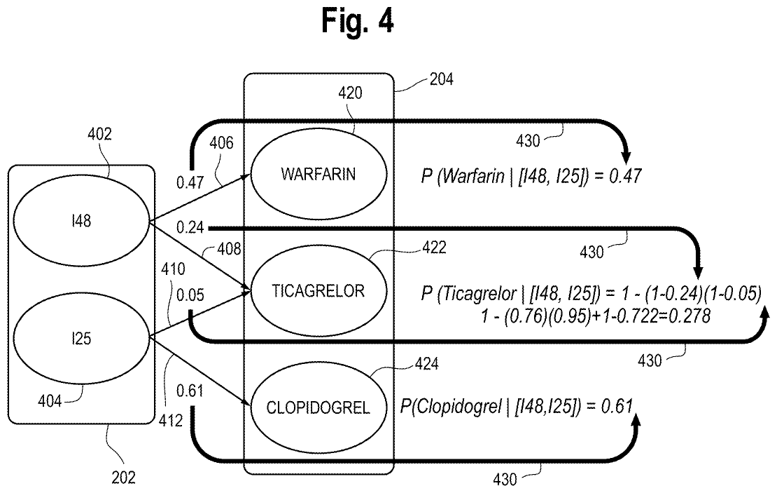

FIG. 4 is another illustration of a hypothetical Bayesian network of FIG. 2 for a simple example of two diseases represented by ICD-10 codes I48, I25 (402, 404), in the Disease Status partition 202 and three medications (Warfarin, Ticagrelor, Clopidogrel) 420, 422, 424 in the Medications partition, showing examples of the calculation of P(M) in accordance with Equation 1. The arrows 430 are provided to show where the weights of the links 406, 408, 410, 412 are inserted in the calculations for P(M). The links between the medications and genetic variants in a genetics partition (206, see FIGS. 2 and 3) are not shown, in this example each medication has a link to at least one genetic variant with a binary value of 1.

The indication of PGx relevance comes from having an above-threshold probability for any of the specific medications that have PGx relevance. It is possible to generate a single value to represent their probability of benefiting from PGx testing, especially Equation (1) integrates more information such as the likelihood of allele presence. Alternatively, the value could be represented by the calculation for the probability P of medication prescription: Equation (2) P=1−(πᵢ1−P(Mᵢ)),Mᵢ ϵ M, where M is the set of all medications for which the patient has an above-threshold probability estimate. P could be the patient's PGx score, essentially.

The probability of each medication is calculated given the disease profile available for that patient. The probabilities for Warfarin being prescribed and Clopidogrel being prescribed, given the disease profile of I48 and I25 are fairly straightforward, as they have only one link. The probabilities are thus the single weight of the edge connecting I48 to Warfarin and I25 to Clopidogrel. For Ticagrelor, the probability is the combination of probabilities of either I48 or I25 or both resulting in Ticagrelor being prescribed. The prescriptions are assumed to be independent, so they can be calculated through a product of the events. P(Ticagrelor|[I48, I25])=P(Ticagrelor|I48)*P(Ticagrelor|I25)+P(Ticagrelor|I48)*P(No Ticagrelor|I25)+P(No Ticagrelor|I48)*P(Ticagrelor|I25). An alternative method for performing the calculation is that the result is just the compliment of both disease diagnoses not resulting in a Ticagrelor prescription: P(Ticagrelor|[I48, I25])=1−P(No Ticagrelor|I48)*P(No Ticagrelor|I25)=1−(1−0.24)(1−0.05)=1−(0.76)(0.95)=1−0.722=0.278. If there were more disease diagnoses present that result in a Ticagrelor prescription, the complimentary probability of that linkage (p) would just be added into the product [1−(0.76)(0.95)(1−p)].

### Validation

Primary validation of MAPPeR involved assessment of the performance of the disease-to-medication mapping (i.e., the links and weights between the disease status partition 202 and the medications partition 204, FIG. 2), as this is the only predictive aspect of the Bayesian network. Three phases of validation were undertaken to assess the performance. First, an investigation of the internal consistency of each link between diseases and medications was assessed using a binomial hypothesis test. The patient data was partitioned into five subsets, followed by five-fold construction of the disease-to-medication mapping by iteratively leaving out one of each of the five subsets. Each constructed mapping contained over 700,000 links between diseases and medications. The aforementioned binomial hypothesis test was performed on each link from each mapping using the left-out partition as a test set. In each of the five assessments, over 99% of the links were determined to be a reasonable representation of the probability using a 0.05 significance threshold.

Second, a similar binomial hypothesis-based approach was used to assess the performance of the mapping system on external data. The disease-to-medication mapping was constructed using the entire set of patient data and tested against a set of 170 records from patients in the VA Precision Oncology Cohort A, which was obtained as part of the VA's Al Tech Sprint. External consistency was determined similarly as the internal consistency, with a binomial hypothesis test performed for each of the links between disease and medication. In this assessment, 97.4% of the links were found to be consistent between the mapping and VA patient data.

Lastly, a rigorous assessment of the disease-to-medication mapping was carried out by varying the operational threshold used to define a reliable mapping from disease diagnosis to medication prescription. For this evaluation, operational thresholds of the posterior probability were varied from 0 to 1, which resulted in an Area Under the Receiver

Operating Characteristic Curve (AUC) of 0.737. An optimal operational threshold identified as that closes to perfect performance (Sensitivity=1.0, Specificity=1.0) was determined to be 0.01, corresponding to a 0.562 sensitivity, 0.889 specificity, and 10.259 Diagnostic Odds Ratio. Thus, this threshold 0.01 can be used to make the recommendation of whether or not the patient is likely to benefit from a PGx test, where if the probability P(M) is at or above the threshold the test is recommended.

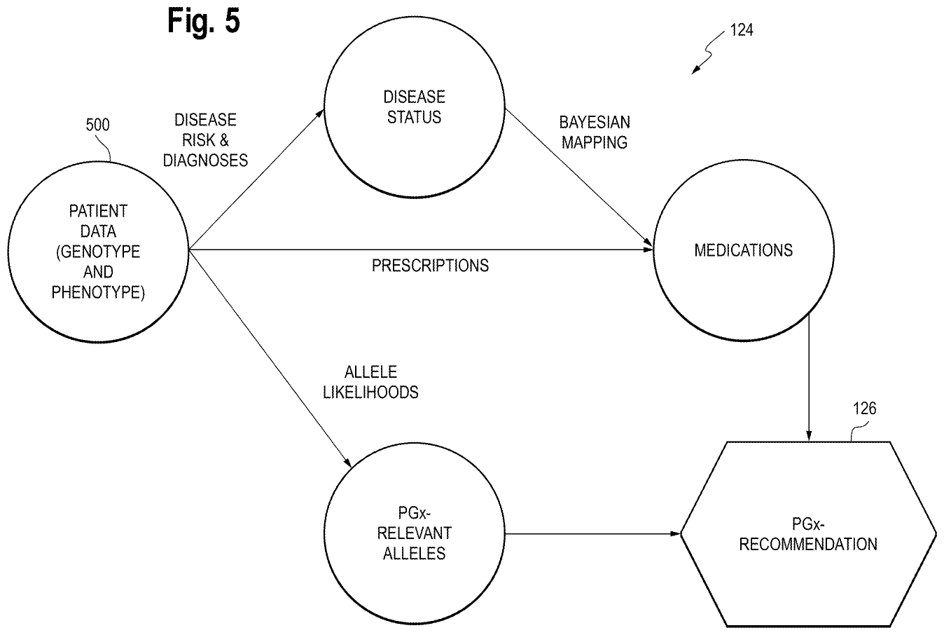

It is possible to add additional layers of predictive value to our method to make it more sensitive to potential impact of PGx testing and guide the end user in a more nuanced way. For example, we could factor in not just existing ICD-10 codes for existing diseases but also disease risk calculations based on any other relevant clinical data. This would add even more pre-emptive value by noting the likelihood of needing a medication before a disease state has even arisen. Another example would be to weight the likelihood of having an actionable genetic variant based on the prevalence of that genetic variant in the population. Thus, PGx testing may be more or less likely to bring value if the genetic variant in question for a particular drug is more or less prevalent. Disease risk prediction would provide a little more predictive value to the disease status information, i.e. we could preemptively determine what diseases they may experience in a given time frame. The allele likelihoods would not be integrated into the Bayesian network, but they would also help determine the PGx recommendation, in conjunction with predicted medications. For PGx to be useful, a patient needs to be prescribed particular medications and have specific allele(s) related to medication. These two things together will make the results of a PGx test useful. The allele likelihoods would be connected to the predicted medications to make a recommendation. Essentially, a patient might be predicted to be prescribed Codeine, but unless there is some degree of certainty they will have a CYP2D6 variation, the PGx test would not be all that useful. FIG. 5 represents a flowchart for integrating all these pieces of information, including patient data 500 (genotype and phenotype), the extraction of disease status, Bayesian mapping between disease status and medications (FIG. 2) and prediction of PGx relevant alleles based on the patient data 500. The Bayesian Network is part of the overall framework, going from disease status to medication prescription, as explained above, and the predicted PGx —relevant alleles can be factored into an overall PGx testing recommendation 126.

### Further Considerations

Our current implementation of MAPPeR uses a server constructed using a Shiny application user interface. Our planned implementation would include improving the user interface and adding connectivity to CMS Blue Button and VA Health API.

In one configuration, implementation into clinical care would come with integration into the clinical practice at a hospital or medical center, and/or the Veterans Administration (VA) or subdivision thereof. We can integrate the backend of MAPPeR into an electronic medical record and test various approaches to alerting a provider that a patient meets the PGx criteria. In addition to adding layers to make the clinical prediction model more sophisticated, interfacing with the EMR can make the whole process more automated so that a provider could be a more passive participant rather than having to affirmatively enter in a patient's data. Thus, MAPPeR would always be working in the background and only alert a provider when they are actively seeing a patient who would benefit from testing.

FIG. 6 is a flow chart illustrating one possible configuration for method of selectively conducting pharmacogenomics testing on a multitude of patients of a healthcare provider. At step 602 input data for each of the multitude of patients is obtained, for example by pulling ICD-10 codes for all patients in in the healthcare provider system from the EMRs. At step 602, the MAPPeR test is performed as explained above in the context of FIGS. 1 and 2 for each of the multitude of patients based on the input data. At step 606, the probabilities P(M) for each of the patients are used to prioritize which patients should be subject to pharmacogenomics testing, e.g., by comparing the probabilities to a threshold, such as 0.01. At step 608, PGx testing for the patients prioritized in step 606 is performed. As indicated by the loop 610, the steps 602, 604 and 606 is conducted daily, and new patients prioritized by reiteration of steps 602, 604 and 606 are then subject to PGx testing.

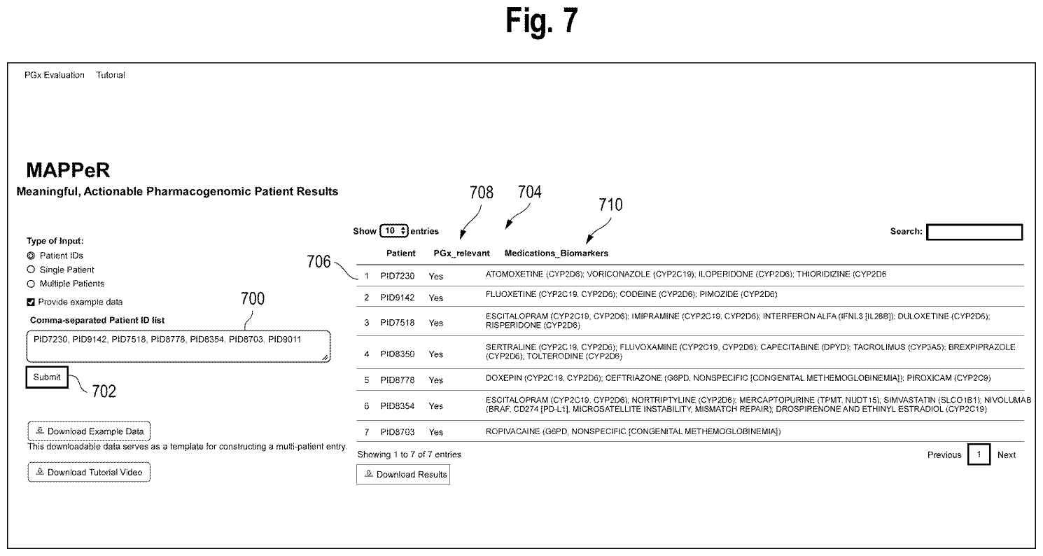

FIG. 7 is an illustration of a front-end user interface showing PGx testing recommendations for a set of patients of a healthcare provider, using the methodology of FIG. 6. In text box 700 a user (e.g., doctor) inputs the patient ID numbers of a set of patients for which they wish to know if they would benefit from PGx testing. In this instance, seven patient ID numbers have been typed into the box 700. The user then clicks on submit 702. The computer of FIG. 1 then obtains diagnostic ICD-10 codes from the electronic medical records of each of the patients and generates a Bayesian network (FIG. 2), executes the method as described previously using the network and generates an output, in this case a prediction of whether the probability P(M) is greater than the threshold. The area 704 of the interface includes a list 706 of each of the patients and in the second column 708 there is text indicating whether or not PGx testing is recommended. In this instance testing is recommended for all 7 patients. In the region 710 of the interface there is a list of the medications and associated PGx biomarkers that are associated with the probability P(M). For example, with patient PID7230, there are four different medications relevant to that patient's disease state (atomoxetine, voriconazole, iloperidone, thioridizine) and each drug is associated with a particular gene or genetic variant, shown in parenthesis next to the drug name. The user perusing the recommendations shown in FIG. 7 can then order PGx testing for any or all of the patients listed by toggling to a new screen or accessing the patient's HER and entering the appropriate order for PGx testing.

As noted in FIG. 1, one possible application is a patient themselves using a user interface (e.g., web browser) that is linked to the computer 120 implementing the Bayesian network and providing tools on the computer 132 (or equivalently smartphone or tablet) such as prompts and displays that allow the patient to determine whether they are likely to benefit from pharmacogenomics testing. For example the computer 132 could include a tool such a text box or the like for the patient to input information as to diseases they have been diagnosed with or medications they have been prescribed, either directly or indirectly. Alternatively the tool could allow the user to insert their name or other unique identifying indicia. The computer includes an applications programming interface linking the information entered in the tool described above and the computer 120, see FIG. 1. The information entered in the tool is converted to the input data set for the Bayesian network implemented in the computer 120. The interface on the computer 132 could include a second tool, such as display screen or text prompt reporting to the patient the recommendation or prediction generated by the computer 120 either directed or indirectly. For example, the second tool could be a text display such as: “Hello [name]. MAPPeR indicates that you would likely benefit from a pharmacogenomics test. Contact your doctor.”

The appended claims are offered as further descriptions of the disclosed inventions. All questions concerning scope are to be answered by reference to the appended claims.

1. A computer-implemented method for determining whether to perform a pharmacogenomics test on a patient, comprising:
obtaining an input data set comprising at least one of data indicative of a disease status of the patient or data indicative of currently prescribed medications for the patient;
implementing in the computer a Bayesian network representable as a tripartite graph having links between three partitions:
(1) a disease status partition having elements representing one or more independent diseases of the patient;
(2) a medications partition having elements representing medications associated with the elements of the disease status partition or with the currently prescribed medications for the patient; and
(3) a genetics partition having elements representing particular genetic variations which have a pharmacogenomics relationship with the elements in the medications partition;
wherein weights of links between the disease status partition and the medications partition are based on an analysis of a corpus of patient data comprising prescribed medications and disease diagnoses, and wherein weights of links between the medications partition and the genetics partition have binary values that depend on whether a pharmacogenomics relationship has been established between the elements of the medications partition and the elements of the genetics partition;
generating from the Bayesian network a probability of the patient being prescribed a particular medication having a pharmacogenomics relationship with one of the genetic variations in the genetics partition, P(M), based on the input data; and
predicting whether the patient will likely benefit from a pharmacogenomics test based on the generated probability P(M).

2. The method of claim 1, wherein the probability P(M) is generated in accordance with equation (1).

3. The method of claim 1, wherein the input data set comprises a set of ICD-10 codes or the equivalent representing diagnoses of independent diseases assigned to the patient.

4. The method of claim 1 wherein predicting whether the patient will likely benefit from the pharmacogenetics test comprises comparing on the probability P(M) to a threshold that is less than or equal to 0.01.

5. The method of claim 1, further comprising generating by the computer a prediction of the risk of the patient developing a disease in a given time frame in the future.

6. The method of claim 5, further comprising predicting medications for the predicted future disease, and generating with the Bayesian network a prediction of the probability that the patient will be prescribed a medication with a pharmacogenomics relationship in the future.

7. The method of claim 6, further comprising assigning a weight to the likelihood of the patient having an actionable genetic variant based on the prevalence of the genetic variant in the general population, and using the weight in the Bayesian network to generate the prediction of the probability that the patient will be prescribed a medication with a pharmacogenomics relationship in the future.

8. An improved computer configured to determine whether to conduct pharmacogenetics testing on a patient, comprising:
a memory storing an input data set comprising at least one of data indicative of disease status of the patient, or data indicative of currently prescribed medications for the patient;
a processing system configured to implement a Bayesian network representable as a tripartite graph having three partitions:
(1) a disease status partition having elements representing one or more independent diseases of the patient;
(2) a medications partition having elements representing medications associated with the elements of the disease status partition or with the currently prescribed medications for the patient; and
(3) a genetics partition having elements representing particular genetic variations which have a pharmacogenomics relationship with the elements in the medications partition;
wherein weights of links between the disease status partition and the medications partition are based on an analysis of a corpus of patient data comprising prescribed medications and disease diagnoses and wherein weights of links between the medications partition and the genetics partition have binary values that depend on whether a pharmacogenomics relationship has been established between the elements of the medications partition and the elements of the genetics partition;
executable instructions for the processing unit to generate from the Bayesian network a probability of the patient being prescribed a particular medication having a pharmacogenomics relationship with one of the genetic variations in the genetics partition, P(M), based on the input data; and
predicting whether the patient will likely benefit from a pharmacogenomics test based on the generated probability P(M).

9. The apparatus of claim 8, wherein the executable instructions calculate the probability P(M) in accordance with Equation (1).

10. The apparatus of claim 8, wherein the input data set comprises a set of ICD-10 codes or the equivalent representing diagnoses of independent diseases assigned to the patient.

11. The apparatus of claim 8, wherein the instructions further include a threshold for recommending a pharmacogenetics test based on the probability P(M) and wherein the threshold is less than or equal to 0.01.

12. The apparatus of claim 8, further comprising instructions for generating a prediction of the risk of the patient developing a disease in a given time frame in the future.

13. The apparatus of claim 12, wherein the instructions include instructions for predicting medications for the predicted future disease, and generating with the Bayesian network a prediction of the probability that the patient will be prescribed a medication with a pharmacogenomics relationship in the future.

14. The apparatus of claim 12, wherein a weight is assigned to the likelihood of the patient having an actionable genetic variant based on the prevalence of the genetic variant in the general population, and using the weight in the Bayesian network to generate the prediction of the probability that the patient will be prescribed a medication with a pharmacogenomics relationship in the future.

15. A method of selectively conducting pharmacogenomics testing on a multitude of patients, comprising:
a) obtaining input data for each of the multitude of patients;
b) conducting the method of claim 1 for each of the multitude of patients based on the input data;
c) using the predictions P (M) for each of the patients to select a subset of the multitude of patients to subject to pharmacogenomics testing, and
d) conducting pharmacogenomics testing for the selected subset of patients.

16. The method of claim 15, wherein steps a), b) and c) are performed daily for each patient of the healthcare provider.

17. The method of claim 15, wherein the input data of step a) comprises one of (a) patient medical record number, or (b) patient identifying information such as name, or number associated with the patient.

18. The method of claim 15, wherein the healthcare provider is at least one of a hospital or medical clinic.

19. The method of claim 18, wherein the healthcare provider is the United States Veterans Administration or a subdivision thereof.

20. An apparatus for determining whether to perform pharmacogenomics testing on a patient, comprising:
a user interface comprising a display that is operable by the patient to input information as to diseases they have been diagnosed with or medications they have been prescribed;
a processing system configured to implement an applications programming interface that provides the information entered in the user interface to the computer of claim 8, wherein the information entered in the toot user interface is converted by the applications programming interface to the input data set for the computer,
wherein the user interface is also configured to report, to the patient, the prediction generated by the computer.
